(19) United States
(12) Patent Application Publication
(10) Pub. No.: US 2011/0230768 A1
(43) Pub. Date: Sep. 22, 2011

(54) METHOD AND DEVICE FOR PLANNING AND PERFORMING A BIOPSY

(75) Inventors: Dror Nir, Waterloo (BE); Rina Nir, Waterloo (BE)

(73) Assignee: ADVANCED MEDICAL DIAGNOSTICS HOLDING S.A., Waterloo (BE)

(21) Appl. No.: 13/131,553

(22) PCT Filed: Dec. 15, 2008
(86) PCT No.: PCT/EP08/67525

§ 371 Date: May 26, 2011

(51) Int. Cl. A61B 8/13; A61B 10/02

(52) U.S. Cl. 600/461

(57) ABSTRACT

The present invention relates to a method for planning an ultrasound guided biopsy of at least one region of interest in an organ comprising the steps of: a) obtaining backscattered 2D or 3D ultrasound data of a region of the organ, b) identifying one or more regions of interest in said three-dimensional volume using a first processor configured to analyze said 2D or 3D volume data, c) selecting in said first processor the characteristics of at least one ultrasound transducer suitable for the guidance of the biopsy comprising a biopsy needle, d) registering the transducer's characteristics selected in step (c) with the identified region of interest of step (b) using said processor, e) generating at least one biopsy scan plane image using the results of the registering step (d); and f) indicating on said simulated scan plane image the identified region of interest and at least one simulated needle trajectory. The present invention also relates to a method for performing ultrasound guided biopsy.

### FIELD OF THE INVENTION

The present invention relates to a method and apparatus for planning a biopsy. The present invention also relates to a method for performing ultrasound guided biopsy of at least one region of interest in an organ.

### BACKGROUND OF THE INVENTION

When biopsies are performed for the purpose of diagnosing cancer, it is important to correctly target the biopsy procedure such that a “biopsy sample” is taken from the cancer lesion as such.

Cancer is the most commonly diagnosed disease in human. When diagnosed at an early stage, the disease is curable, and even at later stages treatment can be effective. However, once the tumor has extended beyond the region of origin, the risk of metastases increases.

It is therefore important to diagnose clinically relevant cancers at a curable stage. Definitive diagnosis of cancer involves the detection of cancerous tissue obtained from the organ during biopsy.

Ultrasound-guided biopsy methodologies such as for the detection of prostate or breast cancer are well-known and require needles to be inserted into the body to obtain a biopsy sample of one or more target tissue areas. While US guided biopsy has become a commonly-performed procedure, it is not without limitations and controversy.

For example, in the diagnosis of prostate or breast cancer, a region of the organ is imaged using ultrasonic radiation. The practitioner obtains an image of the region of the organ and then, based on the image, selects a site in the organ from where a biopsy is to be obtained. A cannula is then introduced into the organ to the site and a biopsy is obtained.

In this method of obtaining biopsies, it is difficult to correctly target the site of interest. The inability to accurately determinate the site of interest results in biopsies not being obtained from sites where a biopsy should have been obtained, resulting in a false negative biopsy. A cancer patient, which had biopsy samples taken outside the lesion, will be wrongly diagnosed as not having cancer. The end result is patients harboring cancer at early and curable stages. Management of these patients, as well as those diagnosed with early stage disease, has generated a great deal of debate and controversy, driving the need for improved biopsy techniques.

There is therefore a need to develop new methods for improving the targeting of suitable biopsy sites. It is accordingly, an object of the present invention to provide a novel method and system for planning and performing a biopsy on a region of interest. It is also an object of the present invention to overcome or ameliorate at least one of the disadvantages of the art, or to provide a useful alternative thereto.

### SUMMARY OF THE INVENTION

The present invention therefore concerns a method and system for planning an ultrasound guided biopsy of at least one region of interest in an organ said method comprising the steps of:

a) obtaining backscattered 2D or 3D ultrasound data of a region of the organ;
b) identifying (screening for) one or more regions of interest in said three-dimensional volume using a first processor configured to analyze said 2D or 3D volume data;
c) selecting and/or entering in said first processor the characteristics (for example the model and/or the type) of at least one ultrasound transducer suitable for the guidance of the biopsy comprising a biopsy needle,
d) registering the transducer's characteristics selected in step (c) with the identified region of interest of step (b) using said processor,
e) generating at least one biopsy scan plane image using the results of the registering step (d); and
f) indicating on said simulated scan plane image the identified region of interest and at least one simulated needle trajectory.

According to an embodiment, the method also comprises the step of adding anchor information on said biopsy scan plane, wherein said anchor information are selected from the group consisting of the border of the organ, landmarks, bone features, calcifications, or other tissue morphology landmarks.

The methods and systems described in this application can be applied to any organ to be examined such as the prostate, the breast, the thyroid, the liver, the ovaries, the uterus, or other organs.

The method is preferably performed using a 2D ultrasound biopsy transducer, such as for example a biplane biopsy transducer. For example said ultrasound biopsy transducer can be a transrectal or a transvaginal ultrasound transducer.

In an embodiment, the present method comprises the steps of forming a correlation between the identified region of interest the transducer's characteristics, and a possible needle trajectory. The correlation provides a “simulated virtual ultrasound plane”, which is a plane that can be acquired given the specific transducer, and on which the region of interest is reached by a simulated biopsy trajectory. The needle trajectory as well as the plane should be feasible, given the characteristics of the biopsy transducer and needle position.

The ultrasound biopsy transducer characteristic entered into the processor comprises at least one of the transducer type, the transducer model, the transducer length, the transducer width, the transducer geometry, the geometry of the transducer scan, the needle length, the needle width, or combinations thereof. The characteristic may also comprise data concerning the putative position of the transducer compared to the organ, as well as its insertion path. The insertion path is applicable for transrectal and for transvaginal transducers as well as to some surgical transducers.

The present invention also concerns a system for planning an ultrasound guided biopsy procedure, comprising:

(a) at least one ultrasound transducer configured to irradiate an organ and to detect ultrasound data reflected or transmitted by the organ;
(b) at least one ultrasound biopsy transducer comprising a biopsy needle;
(c) at least one processor configured to
analyze the reflected ultrasound data and identify one or more regions of interest in said reflected ultrasound data;
receive the characteristic of the ultrasound biopsy transducer and needle,
register the transducer and needle characteristics with at least one region of interest,
generate at least one simulated biopsy scan plane image using the registered data.

The present invention also concerns a method for the ultrasound guided biopsy of at least one region of interest in an organ comprising the steps of:

(a) using the method for planning an ultrasound guided biopsy according to the invention for generating a simulated scan plane image indicating therein at least one region of interest and at least one simulated needle trajectory, and
(b) obtaining at least one biopsy sample using an ultrasound biopsy transducer comprising a needle, by following the simulated needle trajectory of step (a).

The present invention also concerns a method for ultrasound guided biopsy of an organ comprising the steps of

i) obtaining at least one simulated biopsy scan plane image of the organ, said image showing biopsy trajectory and at least one region of interest;
ii) performing an ultrasound guided biopsy on said organ using least one ultrasound biopsy transducer comprising a biopsy needle comprising the steps of:
iii) obtaining at least one real time scan plane image using said ultrasound biopsy transducer,
iv) combining the at least one simulated scan plane image with the at least one real time image,
v) positioning the ultrasound biopsy transducer according to the simulated biopsy scan plane,
vi) performing a biopsy, and
optionally repeating step iii) to vi).

Preferably said at least one simulated biopsy scan plane image of the organ is obtained using a method for planning an ultrasound guided biopsy of an organ according to the invention.

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### BRIEF DESCRIPTION OF THE FIGURES

FIG. 1A represents a simulated ultrasound scan plane image of a prostate, wherein the contour of the prostate is shown as well as the region of interest (highlighted) and a simulated needle pathway (broken line).

FIG. 1B represents a real time ultrasound scan image of a prostate, wherein the needle shooting distance (broken line) is shown.

FIG. 1C represents the fused image of the simulated image of FIG. 1A and the real time ultrasound scan image of FIG. 1B.

### DETAILED DESCRIPTION

The present invention provides methods and systems for tissue characterization based guided biopsies.

The methods and systems described in this application can be applied to any organ to be examined, preferably to any organ that can be evaluated by ultrasound and where ultrasound guided biopsy is being used. Preferably said organ is selected from the group comprising the prostate, the breast, the thyroid, the liver, the ovaries, the uterus, and the like. In a preferred embodiment, the organ is a prostate or a breast.

Preferably, the method of the present invention comprises several steps.

The first step concern the ultrasound acquisition: obtaining backscattered 2D or 3D ultrasound data of a region of the organ. 2D or 3D ultrasound is performed and backscattered Ultrasound data are acquired. Preferably a 3D volume of backscattered ultrasound data are acquired. It is also possible to acquire sequences of 2D images which are then treated as a 3D bulk. This is referred as “free hand 3D”. This ultrasound acquisition step can be performed using either a 3D transducer or a sequence of images obtained using a 2D transducer.

The second step comprises the tissue characterization and identification of at least one region of interest: The acquired ultrasound data are analyzed by a tissue characterization processor, and at least one or more regions of interest can be detected. Preferably, the Ultrasound data can be analyzed by a processor which is configured to determine locations suspected of having a malignant behavior, as described hereunder.

The processor can be any commercially available central processing unit (CPU) that is enabled by means of a commercially available operating system or a specially developed one to apply the characterization algorithms and other algorithms (e.g. correlation algorithms) on the backscattered Ultrasound data. Following this application, specific mathematical features corresponding to the morphology of the underlying tissue are extracted. The characterization algorithms can be based on computing features like entropy, FFT parameters, wavelets parameters, correlation measures, and are tuned to quantify the probability of the analyzed tissue to be categorized as malignant or non-malignant tissue.

In an embodiment, the characterization algorithms are selected from the group comprising a Fourier analysis, a wavelet analysis and an entropy analysis. The characterization algorithms are designed to detect different tissue pathologies. The characteristic features that are related to a predetermined condition like healthy tissue, and those characteristic features that are related to a predetermined condition like malignant tissue are identified. The meaning of “designed” is the identification and selection of those features that are best separating the two pathological phenomena.

Suitable characterization algorithms for identifying the region of interest are sufficiently sensitive to changes in the backscattered energy induced by the alterations in the tissue morphology typical of the disease to be detected. Suitable characterization algorithms are described in U.S. Pat. No. 6,785,570 and PCT application WO 2004/000125 the subject matter of which is incorporated herein by reference. A suitable program for the characterization is the characterization software Histoscanning™ (Advanced Medical Diagnostics, Waterloo, Belgium).

The next step comprises the planning step as such: the planning step starts once a tissue characterization analysis has been done. The tissue characterization should not necessary be full, but may be confined to the organ, or to a tissue area that is manually identified by a person (partial characterization). If some suspicious regions of interest are identified then the next step is to plan which one to target by biopsy. The biopsy is therefore being planned based on the results of characterization step 2.

This step results in zero, in case no lesion are found, for example, or one or more preplanned needle insertions. This planning stage can be a semi automatic process where the user is assisted in defining the optimal scan plane.

The selection of regions of interest to be biopsied can be user dependent or can be selected by the processor. In this step, the targeted lesions or regions of interest are selected based on their size, their distance from the transducer and/or their accessibility to biopsy (some lesions may be found in areas which are easier to reach by a biopsy needle) and/or their estimated aggressiveness (preference to more aggressive lesions). Preferably, bigger size regions of interest (lesions) are targeted. Preferably, accessible regions are selected. Regions that are not marked as suspicious can also be selected depending on the practitioner experience or medical guidelines.

The targeting can be done by the practitioner or by the processor based on the above described criteria.

The planning step also comprises the acquisition of information regarding the details of the planned biopsy procedure The planning step takes therefore into account the characteristics of the ultrasound biopsy transducer such as the type and model of the transducer and of the needles that will be used, such as for example the, shooting distance, the shooting angle, etc. Preferably, the ultrasound biopsy transducer performs 2D scans.

The process then registers the data concerning the transducer and the identified regions of interest and determines the suitable shape and angle of the 2D scans to be acquired during the biopsy session in relations to the 3D ultrasound data that was acquired in the first step.

For each of the selected target lesions the method then searches and generates an optimal simulated scan plane which is displayed as an image.

Preferably the scan plane is a plane that can be acquired provided that the transducer will be located on a feasible position on the patient. For example, in the case of transrectal prostate ultrasound, the simulated scan needs to assume that the transducer will be provided in the intended location, and that the geometry of the scan will be correct. Many scans can be acquired depending on the way that the transducer will be rotated.

The scan plane image also shows at least one optimal anticipated needle trajectory. This trajectory takes into account the given needle geometry and the amount of actual suspicious tissue to be sampled. The scan plane image helps the practitioner to visualize how the organ looks and where the region of interest is located and how the biopsy needle can be inserted. Optionally, markers may be indicated on the image, which will help the alignment with real time data. Said markers can be selected from the group consisting of the border of the organ, landmarks, bone features, calcifications, or other tissue morphology landmarks.

The next step comprises initiating the biopsy procedure. The practitioner first inserts a biopsy needle on the ultrasound transducer and starts an ultrasound guided biopsy.

The biopsy procedure also include a guidance step: During this step the practitioner uses the ultrasound biopsy transducer to direct the biopsy needle towards the region of interest, the real time ultrasound image acquired during this step is combined (fused or aligned) with the simulated scan plane image comprising the planned needle trajectory. This step helps the practitioner to position the transducer and the needle in the optimal location determined in the planning step.

The next step in the biopsy procedure is to perform a biopsy once the needle is in the optimal position.

In case of several planned biopsies, the steps in the biopsy procedure can be repeated for the required number of times.

The present invention also concerns a system for planning an ultrasound guided biopsy procedure, comprising:

(a) at least one ultrasound transducer configured to irradiate an organ;
(b) at least one ultrasound transducer configured to detect ultrasound data reflected or transmitted by the organ; This ultrasound transducer is generally the same as the ultrasound transducer configured to irradiate the organ,
(c) at least one ultrasound biopsy transducer comprising a biopsy needle; This ultrasound transducer can be the ultrasound transducer described in (a) which is fitted with a biopsy needle,
(d) at least one processor configured to
analyze the reflected ultrasound data and identify one or more regions of interest in said reflected ultrasound data;
receive the characteristic of the ultrasound biopsy transducer and needle,
register the transducer and needle characteristics with the at least one region of interest, and
generate at least one simulated biopsy scan plane image using the registered data.

For example, the processing workstation (hereby named workstation or processor) is used in combination with a separate ultrasound machine that is connected to it.

In this embodiment, the above mentioned biopsy method steps can performed as described herein using the following steps:

1) Ultrasound acquisition/performed on the ultrasound machine. The data are transferred to the processor configured to analyze the acquired data. The analyzing program can be any computer-aided diagnostic program for Ultrasound data such as Histoscanning™.
2) Tissue characterization is performed and then displayed on a display connected to the processor.
3) the planning step is performed as described above using the processor. Images of simulated biopsy scan plane are obtained.
4) Initiate the biopsy: Using the ultrasound machine that is still connected to processor and streaming the real time image data continuously to the processor.
5) Guidance: The combined image (aligned simulated and real time image) is displayed on the display.
6) Biopsy: The biopsy is performed using the biopsy needle following the planned (simulated) ultrasound biopsy path determined during the planning step.
7) optionally, the above mentioned steps are repeated depending on the number of biopsy sample to be obtained, and the number of regions of interest.

### EXAMPLE

### Example 1

### Prostate Biopsy

The practitioner acquires a 3D ultrasound scan of the prostate of a patient using an ultrasound transducer A. The result is displayed as a 3 orthogonal 3D planes as well as in virtual 3D box that contains the imaged prostate, on which the results of the tissue characterization analysis are displayed. A lesion is then identified on the left lobe close to the rectal wall. The lesion is then identified automatically or by the doctor as the target lesion (the region of interest). For the biopsy the practitioner uses a linear transducer B comprising a needle that has a reach of 3 cm and is of an angle of 45 degrees to the transducer. The planning stage comprises a review of the different scans that the practitioner can make by having transducer B in place and rotating it. The planning stage also takes into account various insertions depths so that the biopsy needle will have the possibility to reach the identified region of interest.

Once the optimal simulated scan plane is identified and fixed, anchor information can then be marked on it. The anchor information usually includes elements which will allow the practitioner to identify during the biopsy session that the right scan position is reached. The anchor information may include for example the borders of the prostate. In addition to the anchor information, the other two items that are highlighted on the planned image are the target lesion and the anticipated needle trajectory.

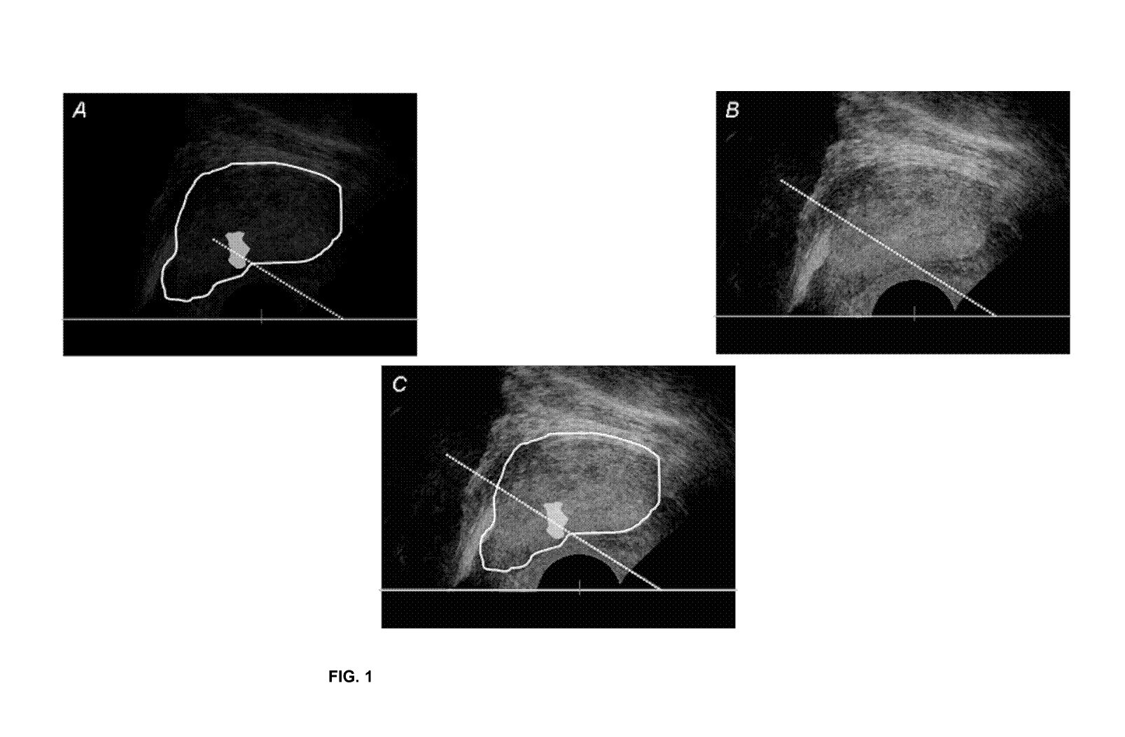

At the end of the planning stage the practitioner can have generated one or more optimal simulated scan planes, with the anchor, lesion, and needle trajectory highlighted as shown in FIG. 1A. The practitioner can arrange these scan planes based on the desired anticipated optimal sequence of biopsy.

During the biopsy stage, the practitioner performs a real time scan to obtain a real time scan as shown in FIG. 1B. The practitioner can go through each of the planned needle insertions and arrive to a real life scan that is as close as possible to the optimal simulated scan plane. To support the practitioner during this stage the real life scans are then visually combined with the simulated one as shown in FIG. 10. This combined visualization is achieved, for example, by:
Displaying the real life scan in one color shades (for example gray levels), while the simulated one is being displayed in other color shades. The best image will be obtained in this situation only when the two scans are aligned on top of each other.
Using the anchor items and the needle trajectory as highly visible references.

While moving the transducer, the fused image is continuously updated and guides the practitioner for the correct alignment of the real life scan situation with the planned optimal simulated scan plane.

Once a needle has been inserted, the real trajectory of the needle can also be compared to the planned trajectory and feedback on its accuracy is given to the practitioner. In case of inaccurate needle insertion the practitioner may decide to continue to try and reach the current target, or he may decide to move on to the next one. When the needle is accurately inserted and a biopsy taken, the user can then proceed with the next target lesion, if any other was planned.

1. A method for planning an ultrasound guided biopsy of at least one region of interest in an organ comprising:
a) obtaining backscattered 2D or 3D ultrasound data of a region of the organ;
b) identifying one or more regions of interest in said three-dimensional volume using a first processor configured to analyze said 2D or 3D volume data, and configured to determine locations suspected of having a malignant behavior, using characterization algorithms;
c) selecting in said first processor the characteristics of at least one ultrasound transducer suitable for the guidance of the biopsy comprising a biopsy needle, wherein the transducer characteristics comprise at least one of the transducer type, the transducer model, the transducer length, the transducer width, the transducer geometry, the geometry of the transducer scan, the needle length, the needle width, or combinations thereo];
d) registering the transducer's characteristics selected in (c) with the identified region of interest of (b) using said processor;
e) generating at least one biopsy scan plane image using the results of the registering (d); and
f) indicating on the simulated biopsy scan plane image of (e) the identified region of interest and at least one simulated needle trajectory.

2. The method according to claim 1, further comprising adding anchor information on said biopsy scan plane, wherein said anchor information is selected from the group consisting of the border of the organ, landmarks, tissue morphology, calcifications, and bone features.

3. The method according to claim 1, wherein said organ is selected from the group consisting of prostate, breast, thyroid, liver, ovaries and uterus.

4. The method according to claim 1, wherein said ultrasound biopsy transducer is a 2D ultrasound biopsy transducer.

5. The method according to claim 4, wherein said 2D ultrasound biopsy transducer is a biplane biopsy transducer.

6. The method according to claim 4, wherein said ultrasound biopsy transducer is a transrectal or a transvaginal ultrasound transducer.

7. The method according to claim 1, further comprising forming a correlation between the identified region of interest and the transducer's characteristics and a possible needle trajectory.

8. (canceled)

9. A system for planning an ultrasound guided biopsy procedure, comprising:
(a) at least one ultrasound transducer configured to irradiate an organ and to detect ultrasound data reflected or transmitted by the organ;
(b) at least one ultrasound biopsy transducer comprising a biopsy needle; and
(c) at least one processor configured to
analyze the reflected ultrasound data and identify one or more regions of interest in said reflected ultrasound data;
receive the characteristic of the ultrasound biopsy transducer and needle;
register the transducer and needle characteristics with the at least one region of interest and
generate at least one simulated biopsy scan plane image using the registered data.

10. A method for the ultrasound guided biopsy of at least one region of interest in an organ comprising:
(a) using the method of claim 1 for generating a simulated scan plane image indicating therein at least one region of interest and at least one simulated needle trajectory; and
(b) obtaining at least one biopsy sample using an ultrasound biopsy transducer comprising a needle, by following the simulated needle trajectory of (a).

11. A method for ultrasound guided biopsy of an organ comprising:
i) obtaining at least one simulated biopsy scan plane image of the organ, said image showing biopsy trajectory and at least one region of interest;
ii) performing an ultrasound guided biopsy on said organ using at least one ultrasound biopsy transducer comprising a biopsy needle comprising:
iii) obtaining at least one real time scan plane image using said ultrasound biopsy transducer;
iv) combining the at least one simulated scan plane image with the at least one real time image;
v) positioning the ultrasound biopsy transducer according to the simulated biopsy scan plane;
vi) performing a biopsy; and
optionally repeating iii) to vi).

12. The method according to claim 11, wherein said at least one simulated biopsy scan plane image of the organ is obtained using a method according to claim 1.

13. The method according to claim 10, wherein said organ is selected from the group consisting of prostate, breast, thyroid, liver, ovaries and uterus.

14. The method according to claim 10, wherein said ultrasound biopsy transducer is a 2D ultrasound biopsy transducer.

15. The method according to claim 14, wherein said 2D ultrasound biopsy transducer is a biplane biopsy transducer.

16. The method according to claim 14, wherein said ultrasound biopsy transducer is a transrectal or a transvaginal ultrasound transducer.
